(19) United States
(12) Patent Application Publication
(10) Pub. No.: US 2024/0053238 A1
(43) Pub. Date: Feb. 15, 2024

(54) SUBLIMATING SYSTEM

(71) Applicant: LUXEMBOURG INSTITUTE OF SCIENCE AND TECHNOLOGY (LIST), Esch-sur-Alzette (LU)

(72) Inventors: Veneranda LOPEZ DIAS, Esch-sur-Alzette (LU); Christophe HISSLER, Thionville (FR); Francois BARNICH, Bastogne (BE); Julian KLAUS, Bonn (DE); Laurent PFISTER, Belvaux (LU); Olivier DE CASTRO, Niederkorn (LU); Hung Quang HOANG, Esch-sur-Alzette (LU); Rachid BARRAHMA, Soleuvre (LU)

(21) Appl. No.: 18/254,422

(22) PCT Filed: Nov. 24, 2021
(86) PCT No.: PCT/EP2021/082753

§ 371 Date: May 25, 2023

(30) Foreign Application Priority Data
Nov. 25, 2020 (LU) LU102231

(51) Int. Cl. G01N 1/42; G01N 33/00

(52) U.S. Cl. CPC G01N 1/42; G01N 33/0011

(57) ABSTRACT

A system and method for sublimation of water ice and/or water ice from a regolith. The system comprises a sublimation chamber under high to ultra-high vacuum into which protrudes a shield of a thermal management system. Sublimated water is collected to be further analysed or analysed in-line. For instance, cold traps can be used to trap water and transfer it to sample tube for analysis.

### CROSS-REFERENCE TO RELATED APPLICATIONS

The present invention is the US national stage under 35 U.S.C. § 371 of International Application No. PCT/EP2021/082753 which was filed on Nov. 24, 2021, and which claims the priority of application LU102231 filed on Nov. 25, 2020 the contents of which (text, drawings and claims) are incorporated here by reference in its entirety.

### FIELD

The invention relates to experimental devices for analysing the content of a sample and more particularly to sublimate gas out of a solid sample and/or to sublimate a sample itself, under vacuum and low to cryogenic temperature.

### BACKGROUND

A cryogenic extraction system is discussed in Lecuyer et al. (“D/H fractionation during the sublimation of water ice”, 2017, DOI:10.1016/j.icarus.2016.12.015). This system consists in a set-up of a glass vacuum line wherein a resistance heating wire is coiled around the glass pipes and a temperature controlled cryo-trap aiming at the study of the isotopic fractionation during pure ice sublimation process. This cryo-trap comprises a heating wire arranged around a glass tube isolated from the liquid nitrogen bath by an envelope of helium gas within a Pyrex™ chamber. Two tubes that are submerged into LN2 are used to collect the sublimated water. This system does not enable a pressure lower than 10⁻⁵ mbar. The narrow connections between the glass pipes and the vacuum system result in an insufficient gas evacuation rate, which leads to a partial deposition of the sublimated water on the ice surface. This fact decreases the sublimation rate and modifies the expected kinetic isotope fractionation, since other process will happen in addition at the ice surface. Moreover, the pressure varies and cannot be properly controlled. The temperature is not homogeneous along the entire pipe system and it cannot be fully controlled. The experiments are thus hardly reproducible.

In addition, this set-up does not admit the connection of a sample transfer system when the sample is prepared under lunar environmental conditions. Hence, the sample needs to be exposed to Earth environmental conditions during its introduction into the set-up. Finally, since the frozen sample is transferred to the sublimation element by heating and refreezing it, the initial ice structure will be modified, and it is not suitable for dusty ice and/or icy regolith.

There is therefore a need for an improved sublimating system.

### SUMMARY

The invention aims at providing a sublimating system without the drawbacks mentioned above and in particular a system which enables at least one of: a high to ultra-high vacuum, low to cryogenic temperatures, no deposition of water within the sublimating system, a high gas evacuation rate and a better thermal control of the experiment.

The invention relates to a system for sublimating a sample under high to ultra-high vacuum and at low to cryogenic temperature, and for collecting or in-line analysing sublimated compounds thereby obtained, the system comprising: a sublimation chamber adapted to receive the sample under high to ultra-high vacuum and at low to cryogenic temperature and provided with an outlet for evacuation of sublimated compounds; a collection system and/or an in-line analysing system coupled to the outlet of the sublimation chamber; a pumping system configurated to convey the sublimated compounds from the sublimation chamber to the collection system and/or to the in-line analysing system; a thermal management system comprising a shield protruding into the sublimation chamber and configured to at least partially surround the sample so as to exchange heat with the sample.

The system can also be provided with a manipulation chamber connectable to the sublimation chamber; and a transfer device for releasably handling a sample holder from the manipulation chamber to the sublimation chamber, so that the sample holder can engage the shield.

The vacuum chamber and the pumping system enable to evacuate the gas, while the shield surrounding the sample ensures that the sample is under a controlled temperature.

According to an exemplary embodiment, the collection system comprises a cold trap fluidly connectable to the outlet of the sublimation chamber, the cold trap in various instances being constituted of a U-shape pipe the outer surface of which being partly immerged into a cold or cryogenic medium such as LN2. The cold trap is thus provided to collect sublimated compounds which will be stored temporarily in a solid state before further analysis.

According to an exemplary embodiment, the cold trap comprises a first end connectable to the outlet of the sublimation chamber, potentially via an interfacing structure enabling or preventing the fluidic connection between the sublimation chamber and the cold trap, and a second end connectable to the pumping system potentially via an interfacing structure, and wherein the cold trap, the optional interfacing structures and the sublimation chamber are optionally configured to be maintained under a dynamic high to ultra-high vacuum.

According to an exemplary embodiment, the collection system and the pumping system are configured to ensure a high evacuation rate in the sublimation chamber, so as to collect all the sublimated compounds.

According to an exemplary embodiment, the cold trap comprises two gate valves respectively at the first and second end of the cold trap.

According to an exemplary embodiment, the cold trap is adapted to be disconnected from the sublimation chamber and to be sealed so as to maintain the sublimated compounds under vacuum.

According to an exemplary embodiment, the system comprises at least two cold traps, sequentially connectable to the sublimation chamber. This enables, for instance, to separate compounds having different sublimation temperatures within the vacuum chamber or different forms of water present in the regolith or different water ice fractions sublimating at different time intervals.

According to an exemplary embodiment, the system comprises a transfer device, for instance a transfer rod for handling the sample or a sample holder, wherein the transfer device is configured to move between a retracted position and an inserted position, wherein optionally the sample holder engages the shield when the transfer rod is in its inserted position. This enables the sample to be introduced in the sublimation chamber under vacuum and at low to cryogenic temperature. It also enables to at least partially automatize the manipulation of the sample.

According to an exemplary embodiment, a thermal insulator element is provided to thermally isolate the sample or sample holder from the transfer device, the thermal insulator element being optionally coupled to the sample or sample holder by a bayonet coupler.

According to an exemplary embodiment, the system comprises a manipulation chamber connectable to the sublimation chamber.

According to an exemplary embodiment, the manipulation chamber is positioned relative to the sublimation chamber such that the sample holder can sit in the manipulation chamber when the transfer device is in its retracted position.

According to an exemplary embodiment, the manipulation chamber comprises at least one of: a port for connection with the sublimation chamber; at least one port for a pressure gauge; at least one port for electrical feedthrough; at least a port for a feedthrough, through which the transfer device protrudes; at least one port for sample introduction, for instance a fast entry door, on which a vacuum view window or a optically transparent window can be mounted; at least one port for dry air/nitrogen gas introduction; at least one port for connecting a pumping system for putting the manipulation chamber under high vacuum; optionally, at least one port for further coupling a transfer suitcase.

According to an exemplary embodiment, the sublimation chamber comprises at least one of: a port for the thermal management system; at least one port for a pressure gauge; at least one port for electrical feedthrough; at least one port for sample introduction; at least one vacuum view window or an optically transparent window; a port for connecting a pumping system for pumping out directly the sublimation chamber.

According to an exemplary embodiment, the collection system comprises at least one port for independently venting the collection system, for instance with dry air or nitrogen gas; and/or at least one port for connecting to the pumping system.

According to an exemplary embodiment, the system comprises a sample holder, for instance having axisymmetric shape, for holding the sample in the sublimation chamber.

According to an exemplary embodiment, the sample holder is removably coupled to the shield, such that the sample holder exchanges heat between the shield and the sample, the sample holder in various instances being provided with a peripheral groove for a snap-in and thermal coupling to the shield.

According to an exemplary embodiment, the system comprises an adapter inserted into a recess of the sample holder.

According to an exemplary embodiment, the system further comprises a radiation shield mounted on the adapter or sample holder for protecting the sample from radiative heat transfer.

According to an exemplary embodiment, the sample holder comprises at least one temperature sensor configured to measure the temperature of the sample holder and/or of the sample and/or in the volume delimited by the shield and the sample holder and surrounding the sample.

According to an exemplary embodiment, the shield comprises an aperture to enable the evacuation of gases from within the shield into the sublimation chamber, the system being provided optionally with a radiation shield protecting the sample from radiative heat transfer through the aperture.

According to an exemplary embodiment, the sample holder is removably connected to the transfer device by a bayonet coupler.

According to an exemplary embodiment, the thermal management system comprises: a thermal source of low to cryogenic temperature; a thermal sensor for measuring a temperature at a location of the source; a heating element for heating the source; the shield having a first end in direct contact with the thermal source at a first interface, and a second end adapted to exchange heat by conduction to/from a sample; two thermal sensors arranged on the shield to measure a gradient of temperature; a controller calibrated for controlling the heating element in response to signals from the thermal sensors so as to maintain the gradient of temperature within a pre-determined range; a vacuum sealing feedthrough comprising a thermal insulator element and optionally a flange, the vacuum sealing feedthrough delimiting around the first interface a vacuum sealed volume so that the shield exchanges heat with the thermal source exclusively by conduction and exclusively at the first interface, the thermal insulator element insulating the sublimation chamber and/or the flange from the thermal source and first end of the shield wherein the insulator is configured to separate physically the thermal source from the vacuum chamber, the insulator insulating thermally the walls of the vacuum chamber and optionally the walls of the isolating flange from the thermal source and from the shield.

The shield of the thermal management system is destinated to surround a sample positioned in a chamber that is put under vacuum. Foreseeing the shield and the thermal source as two distinct parts enable to control the temperature by applying heat at a distance from the position where the sample is to be positioned. This distance and the insulator play a respective role in lowering the gradient of temperature applied to the sample and surrounding environment, as well as in the absence of relevantly colder points than the sample within the sublimation chamber. The latter, avoiding the sublimated compounds from being deposited inside the chamber and therefore enabling their collection or in-line analyses.

The controller can be calibrated by also taking into account the temperature measured by additional sensors placed in/on the cold finger, the shield, the thermal insulator, the sample holder, the adapter and the sample or sample tube. Thus, the controller can be calibrated by heating the heat exchange element of the thermal source and/or of the shield, and by measuring the resulting temperature gradient in the sample and surrounding environment is measured. This allows later on (after calibration) to command only the heating element placed in the heat exchange element or together with the heating elements placed on the shield and/or thermal insulator, while ensuring the small gradient of temperature for the sample and its environment.

According to an exemplary embodiment, the thermal source comprises a heat exchange element for instance in the form of a cold finger or a cold plate, the heating element in various instances being arranged inside the heat exchange element.

According to an exemplary embodiment, the heating element is positioned at a location remote from the sublimation chamber.

According to an exemplary embodiment, the shield which surrounds partially the sample is of tubular shape, or part of a tubular shape, or is an elongated profile.

According to an exemplary embodiment, the collection system comprises at least one pressure sensor.

The invention also relates to a method for sublimating water ice from a sample, such as pure water ice, soil, plants, terrestrial and extra-terrestrial regolith/regolith simulant or other porous media, the method being carried out in a system according to any of the above-mentioned embodiments, wherein the temperature gradient is in various instances less than 5 K, for example less than 2 K, in the volume delimited by the shield and the sample holder and surrounding the sample.

According to an exemplary embodiment, the method comprises the following steps: introducing a sample holder with a sample in the sublimation chamber under low to cryogenic temperature and nitrogen or dry air atmosphere; applying high to ultra-high vacuum in the chamber; coupling the sample holder to the shield of the thermal management system; heating the sample while maintaining the sublimation chamber under high vacuum; maintaining a low temperature gradient within the sample and surrounding environment during the sublimation process; maintaining stable pressure and temperature during the sublimation process; extracting, collecting and analysing the content of the sublimated gases during and/or after sublimation.

According to an exemplary embodiment, the pressure in the sublimation chamber and the temperature in the sublimation volume i.e., the shield, the sample holder, the sample adapter, the sample tube and the sample, are regulated to mimic as close as possible airless planetary bodies conditions. For instance, while the airless planetary bodies can be under vacuum of 10⁻⁹ to 10⁻¹² mbar, the experiment can reach 10⁻⁸ mbar. According to an exemplary embodiment, the temperature of the sample and/or the temperature in the thermal management system is monitored and increased step by step.

According to an exemplary embodiment, the temperature gradient within the sample and surrounding environment during sublimation is sufficiently low for preventing the presence of points relevantly colder than the sample within the sublimation chamber.

According to an exemplary embodiment, once the sample holder is coupled to the shield, the transfer device is retracted prior to the step of heating.

According to an exemplary embodiment, the system comprises a plurality of cold traps and at most one cold trap is fluidically connected to the sublimation chamber at a given time.

According to an exemplary embodiment, the system comprises at least one cold trap comprising two gate valves at each respective ends thereof and the method comprises a step of closing the gate valves and transferring the at least one cold trap to a remote location while maintaining its content under high vacuum, where one gate is opened to transfer the content of the cold trap to a vial and/or to analyse it.

According to an exemplary embodiment, the system comprises a plurality of cold traps transferred simultaneously to the remote location where one gate of each cold trap is sequentially opened and the content of each cold trap is transferred to a vial and/or analysed.

The several aspects of the invention ensure to various degrees to enable a high evacuation rate for the sublimated compounds out of the vacuum chamber, a low gradient of temperature along the shield, within the sample and surrounding environment, or in the sublimation volume and a precise control of the temperature of the sample and surrounding environment. The system of the invention also prevents the presence of colder points where gas could deposit, thereby altering the validity of the analyses and/or preventing the sublimated compounds from exiting the sublimation chamber.

The system of the invention also has further benefits such as a simpler and more flexible design, allowing easily to adapt it as a payload to be launched on future robotic space missions.

The system further allows to carry out the experiments efficiently in terms of time and energy consumption.

### DRAWINGS

FIG. 1 is an exemplary schematic illustration of the sublimation system according to the invention.

FIG. 2 is an exemplary cross-section of a detailed design of the thermal management system, according to the invention.

FIG. 3 is an exemplary isometric view of a sample holding system, according to the invention.

FIG. 4 is an exemplary isometric view of a sample tube arranged on an adapter, according to the invention.

FIG. 5 is an exemplary isometric view of the coupling between the shield and the sample holder, according to the invention.

### DETAILED DESCRIPTION

The following examples and drawings are given for illustration purposes only. The invention is not limited by these examples but only by the appended claims. The various parts of the system can have various properties or be embodied in various ways. Each variant of each part of the system can be combined with each variant of any other parts of the system, unless explicitly mentioned otherwise.

The drawings are schematic and not drawn to scale. Some elements of the system are not illustrated, such as for example: elements for assembling the various parts together (flanges, screws, etc.), elements for properly ensuring sealing of various compartments (seals, etc.), or elements for controlling the system (wires, sensors, actuators, valves, safety devices, etc.).

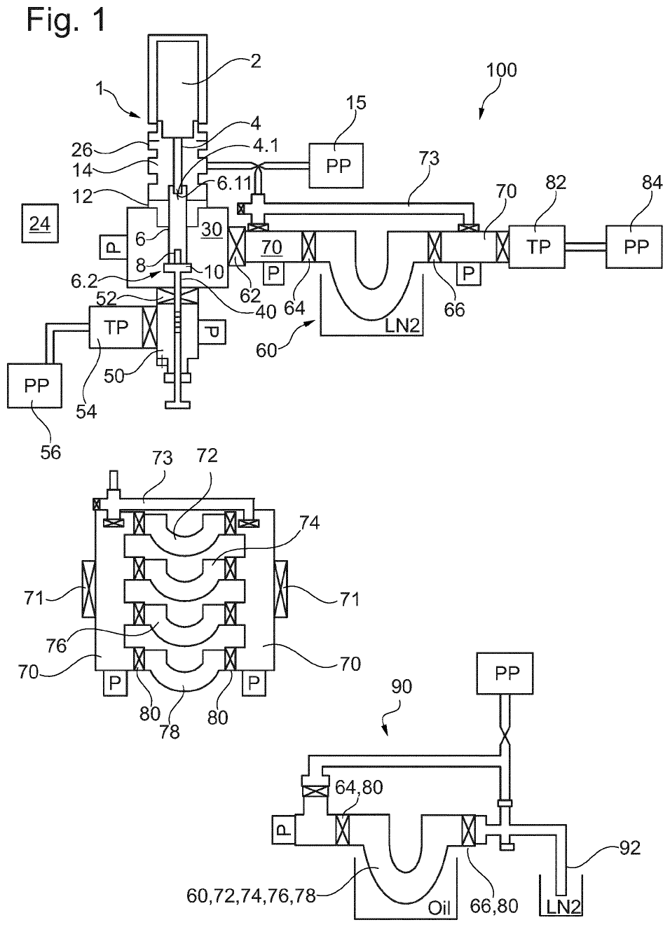

FIG. 1 shows a schematic illustration of a sublimation system 100. The system can comprise a thermal management system 1. The system 1 comprises a thermal source 2, 4 which in an exemplary arrangement is made of a cooling system 2 thermally connected to a heat exchange element 4. The cooling system can be a Dewar containing a cryogenic fluid (LN2, LHe, etc.), a cryostat, a cooling machine, etc. The heat exchange element 4 can be a cold plate, a cold rod, a cold finger, etc.

In one embodiment, the heat exchange element 4 is a cold rod. It can be made of CuBe2 and can be gold plated. It can have an upper conical part connected to the cooling system (e.g., LN2 Dewar).

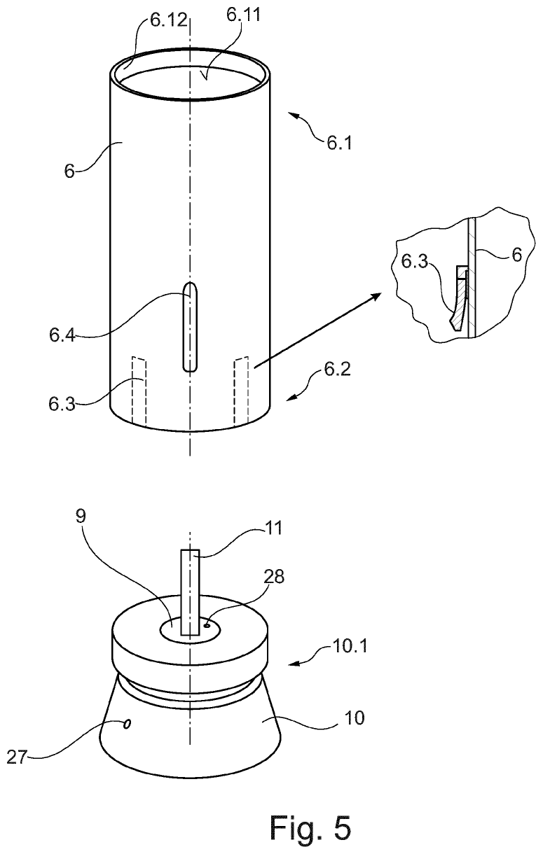

A shield 6 is in direct contact with the heat exchange element 4. The shield 6 has a first end 6.1 (see also FIG. 5) with a surface 6.11 in direct contact with the lower surface 4.1 of the heat exchange element 4. A second end 6.2 of the shield 6, opposite to the first end 6.1 is adapted to exchange heat with a sample 8. The thermal contact between the shield 6 and the heat exchange element 4 happens exclusively at the first surface 6.11. The heat exchange element 4 can have a cylindrical or tubular lower part that is connected with the shield 6.

The shield 6 can be of tubular shape. Alternatively, the shield 6 can have a generally elongated shape with a cross-section that can be partly curved such as an arc of tube, or a closed cross-section such as a polygon, an ellipse or a circle.

The shield 6 can be made of Cu (O free) and/or can be gold plated for increased heat conductivity and for reducing water adsorption. The second end 6.2 of the shield 6 can enable a snap-in rapid coupling of the shield 6 with the sample 8 or with a sample holder 10, for instance through leaf springs and ruby spheres arrangement engaging a groove of the sample holder 10. In an embodiment, the thermal management system is positioned vertically, the first end 6.1 being an upper end, while the second end 6.2 is a lower end. The drawings and part of the description are directed to this vertical orientation. Alternatively, the system 1 can be positioned horizontally or obliquely.

The sample 8 can be a sample tube or any other sample container, provided with a compound to be analysed. The sample can alternatively be self-contained. In exemplary embodiments, the compound is water ice and/or a lunar regolith containing water ice. The sample 8 can be handled together with the sample holder 10.

An insulator 12 is provided to surround at least a portion of the heat exchange element 4. A vacuum sealed volume 14 is defined between the heat exchange element 4 and the insulator 12. Appropriate pumping mechanism 15 and/or valves are provided to ensure a dynamic or static vacuum in the volume 14. The insulator 12 thus ensures the thermal insulation of the heat exchange element 4 from the environment. The insulator 12 also separates physically the cold source 2, 4 from the sample 8.

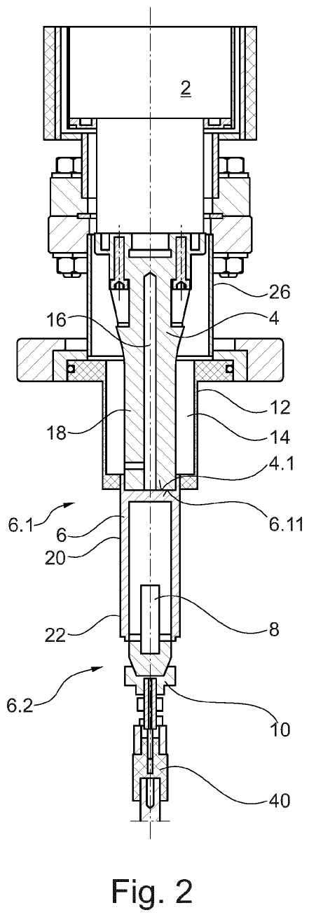

At least one heating element 16 (see FIG. 2) is provided on the heat exchange element 4 to bring the heat exchange element 4 at a higher temperature than the cooling system 2. The heating element 16 can be an electric wire or a heating foil.

The heating element 16 can be arranged internally to the heat exchange element 4 to provide for a more homogeneous temperature at the radially outer periphery of the heat exchange element 4.

The heating element 16 can be positioned at a first half location of the heat exchange element 4, i.e., closer to the cooling system 2 than the shield 6. Optionally, a second heating element (not shown) can be provided at a second half location of the heat exchange element 4. Further heating elements (wires inside the element 4 and/or heating foil outside of it) can be provided at locations closer to the shield 6.

Thermal sensors 18, 20, 22 (see FIG. 2), for example pt100 temperature sensors, are provided on the heat exchange element 4 and the shield 6 to measure the temperature at a location of the heat exchange element 4 and at two locations of the shield, so as to deduce a gradient of temperature over the shield 6. Further sensors can be provided.

When the heating element 16 is complemented with additional heating elements to heat the heat exchange element 4, further thermal sensors can be provided at various locations of the heat exchange element 4.

The sensors transmit measured temperatures to a controller which acts on the heating element 16. The controller is calibrated to control the heating element 16 as a function of the temperatures and gradient of the temperature so as to maintain the gradient within a pre-determined range. This range of temperature can amount to a few K, for example less than 5K, or less than 2K. The calibration is done by empiric learning or by simulation. Based on the temperature from the sensor on the heat exchange element 4 and based on the gradient of temperature measured from sensors on the shield 6, the controller is thus able to determine the amount of energy that the heating element 16 must bring to the heat exchange element 4 (both in terms of power and duration to obtain a steady state).

The shield 6 can also be provided with heating elements (not shown) of the same type as those 16 of the heat exchange element 4. The heating elements on the shield 6 can alternatively be heating foils, attached to the shield by means of gold-plated copper clamps.

The heat exchange element 4 and/or the shield 6 can be gold plated to maximise the heat transfer while preventing water vapour adsorption. This also participates in obtaining a homogeneous temperature distribution throughout the shield 6.

A flange 26 can also be provided. It isolates the lower part of the cooling system 2 from its environment. It can create a vacuum volume around the upper part of the heat exchange element 4, or can be joined to the insulator to create a common vacuum sealed volume around the heat exchange element 4.

According to the invention, a sublimation chamber 30 is provided for receiving the sample under vacuum, high vacuum or ultra-high vacuum. The insulator 12 insulates thermally the heat exchange element 4 and the first end of the shield from the walls of the chamber 30 and or from the walls of the flange, to ensure that the walls do not heat up the heat exchange element 4, or that the heat exchange element 4 do not make some areas of the walls colder. The outer surface of the walls of the chamber can indeed be at room temperature. Likewise, the insulator and optionally together with the flange isolates (in the sense that it separates physically) the cooling system 2 and the heat exchange element 4 from the chamber 30, to ensure that the coldest parts of the thermal management system are not inside the chamber.

This way, the insulator 12 prevents any point within the chamber 30 to be colder than the shield. Thus, during experiments where the sample evaporates or sublimates, the gases do not condensate or deposit on the chamber's walls and/or on the thermal management system elements. This can be particularly advantageous when further analyses are to be done on the gas, as they can be easily evacuated out of the chamber.

For such an experiment, the shield 6 can have an aperture through which the gas can escape from the environment around the sample, so as to be collected and analysed.

The thermal sensors can be arranged below and above the aperture respectively. The surface 6.11 at the interface with the heat exchange element 4 is confined to the vacuum sealed volume 14. The heat exchange element 4 does not protrude out of the insulator 12 or into the chamber 30.

The insulator 12 also isolates the shield 6 from the cooling system 2 and the vacuum in the chamber 30 isolates the shield 6 from the environment. Hence, the shield 6 only exchanges heat by conduction at the surface 6.11 with the heat exchange element 4, and only exchanges heat by conduction at the second end 6.2 with the sample 8 or sample holder 10. The shield 6 exchanges heat by radiation with the walls of the chamber 30—although radiative heat in vacuum is low. The shield 6 protects the sample from such radiation.

Therefore, when the heat exchange element 4 is heated with the wire 16, heat is transferred by conduction to the shield 6, but the cooling system 2 is not in contact with the shield 6 and not reachable by vapours from the sample.

The fact that heat is exchanged by conduction between the cooling system 2, the heat exchange element 4, shield 6 and the sample 8 enables a faster heat transfer than a radiational heat transfer.

The cooling system is first able to cool down the sample because the heat is transferred by conduction from the sample to the cooling system. Then, once the sample is at very low temperatures, heating means warm up the heat exchange element, thereby transferring heat by conduction from the heat exchange element to the sample.

The insulator 12 can be made of polyether ether ketone (PEEK) and constitutes a feedthrough for the shield 6. It can be provided with a heating foil and a pt100 temperature sensor to regulate its temperature (for instance by the controller 24).

For automatically handling the sample holder 10 and the sample 8, the system can be provided with a transfer device 40.

Next to the vacuum chamber 30 (i.e., above, below or aside), a manipulation chamber 50 or a transfer shuttle can be arranged where an operator can manipulate and/or transfer the sample 8. Once the sample 8 is ready for experiments, the transfer device 40 transfers it to the vacuum chamber 30. The transfer device 40, the sample holder 10 and the shield 6 are such that the transfer device 40 brings the sample holder 10 into contact for quick coupling to the shield 6. The transfer device 40 can be a rod actuated by a piston to move up and down and to rotate around its main axis, so as to actuate a bayonet coupling between the rod 40 and the holder 10.

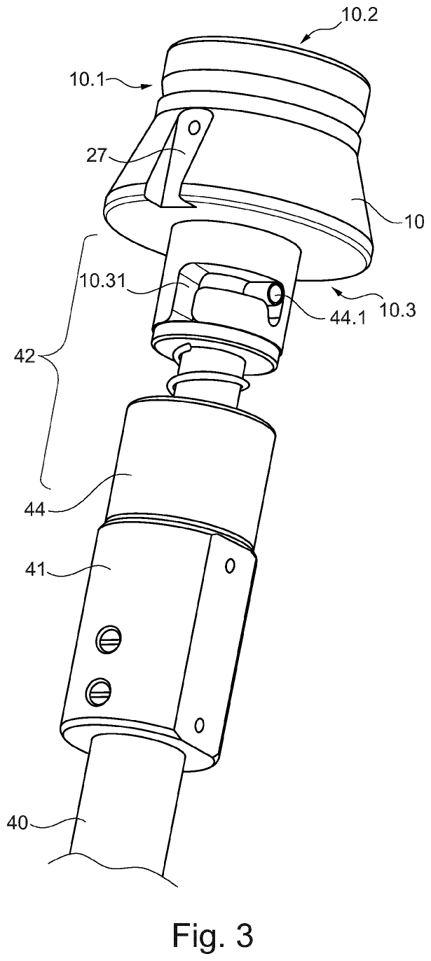

The sample holder 10 and its connection to the transfer device 40 will be further described in relation with FIG. 3.

The sublimation chamber 30 can be equipped with electrical feedthroughs and a pressure gauge and connected to the manipulation chamber 50 by an ultra-high vacuum (UHV) gate valve 52. It is connected to a cold trap 60 through another UHV gate valve 62, which constitutes the outlet for the sublimated compounds out of the vacuum chamber 30. The cold trap 60 can have two opposite ends closable by respective valves 64, 66. As will be discussed below, the sublimation chamber 30 can be connected to a single cold trap 60 or to an interfacing structure 70, which is connected to several cold traps 72, 74, 76, 78, by HV gate valves 80 allowing the collection of different sublimated water vapour fractions during the sublimation process, different forms of water or compounds with different sublimation temperature. The sublimation chamber 30 is continuously evacuated through the cold trap by a turbo pump 82 and a primary pump 84. Sublimation is carried out under dynamic vacuum to ensure the complete removal of the sublimated water vapour from the sublimation chamber 30 at any time. This evacuated water vapour is trapped in the cold traps 60, 72, 74, 76, 78 and does not reach the pumping system 82, 84.

The manipulation chamber 50 is attached vertically to the sublimation chamber 30 through a UHV gate valve 52. This valve 52 is closed during sample introduction into the manipulation chamber 50, which ensures that the sublimation chamber 30 will not be exposed to air, humidity and particles from the ambient. The transfer rod 40 enters the manipulation chamber through a feedthrough from a bottom flange. The manipulation chamber 50 can have a door to enable a fast sample introduction. It can have a port, by which N2 gas is flushed continuously during the sample introduction. This avoids frost formation on the sample tube 8 and on the sample itself.

The introduction procedure can be as follows: the sample is carried from the sample production system to the manipulation chamber in a closed container filled with LN2; this container is opened in the manipulation chamber under continuous N2 gas flushing and the sample tube 8 is introduced into the sample tube adapter 9, or the sample tube and sample adapter 9 is introduced into the sample tube holder 10. Then, the door is closed and the N₂ gas flushing stopped.

The manipulation chamber 50 is connected to a turbo pump 54 and primary pump 56. This pumping system, optionally complemented with the fact that the manipulation chamber 50 is vented with N2 gas and kept into N2 gas during sample introduction, allows to pump out the manipulation chamber 50 in a short time prior to opening the UHV gate valve 52 and transferring the sample to the sublimation chamber 30. This prevents an increase of the sample temperature before it is transferred to the sublimation chamber 30 and before the holder 10 or the sample 8 is attached to the tubular shield 6, which will be at low to cryogenic temperature.

To minimize the increase of temperature in the sample that is manipulated in the manipulation chamber 50, the turbo pump 54 and the primary pump 56 are chosen to obtain rapidly (0.5 to 2 min from atmospheric pressure) a pressure around 10⁻⁵ mbar. Then the valve connecting the turbo pump with the manipulation chamber 50 is closed and the valve connecting the sublimation chamber 30 and the manipulation chamber 50 is opened. The manipulation chamber 50 has an additional valve to introduce nitrogen gas, thus it will help to pump out faster since inert gases are evacuated faster than air containing O2 and moisture (H2O). The sample can for instance be introduced at −195° C. and appropriate pumps are such that the temperature in the sample will be low enough to avoid driving the sample to a temperature greater than −170° C. during sample introduction.

Once the sample tube holder 10 is attached to the tubular shield 6, if the temperature in the sample holder 10 and transfer device 40 do not need to be measured, the transfer rod 40 is detached from the sample tube holder 10 and retracted to the manipulation chamber 50. Next, the UHV gate valve 52 is closed.

The manipulation chamber 50 is equipped with a pressure gauge. It can have a fast entry door provided with an optically transparent view port (glass or quartz), enabling optical measurements of the sample under low pressure and cryogenic temperatures.

In a variant of the system 100, the manipulation chamber 50 is replaced with a manually operated or fully automated interface, enabling to connect a cryo-transport suitcase. The sample is thus carried in this suitcase from the preparation system to the sublimation chamber 30 under low pressure and cryogenic temperatures. The suitcase can be connected to the interface, the interface pumped out into vacuum and the valve connected to the sublimation chamber opened to allow the sample transfer. This system will not need to be flushed with N2 gas during sample introduction in contrast with the manipulation chamber 50 described above, since the sample will be kept in vacuum and cryogenic temperatures from its production to the sublimation chamber 30.

In a variant of the invention (not illustrated), the sublimated compounds are analysed in-line at the evacuation. Such a system can be of simpler design. However, it has some drawbacks: one purpose of the sublimation system of the invention is to study the sublimation process and the related isotope fractionation of water ice under airless planetary conditions, while minimizing the impact of the instrumentation. A direct interaction between the sublimation chamber 30 and the instrument for isotope analyses (laser spectrometer, mass spectrometer) could result in increased isotope fractionation and in inaccurate quantitative analyses of evolved gases. Inaccuracies can result from an inadequate evacuation rate at any time, a non-precise estimation of the system volume, changes in temperature and/or pressure, resulting in an erroneous isotope signature and quantitative analyses interpretation.

In another variant, as shown in FIG. 1, the sublimated compounds are collected by a collection system (60, 70, 82, 84 . . . ). The collection system comprises one cold trap 60 or several cold traps 72, 74, 76, 78 (four cold traps are only an exemplary number). Although other embodiments can exist, in various instances the embodiment is here a U-shape cold trap.

The cold trap(s) can consist in a U-shaped glass tube with a KF (Klein Flansche) flange at both ends to connect both ends via KF compact fast acting high vacuum gate valves 64, 66, 80. The diameter of the U-shape pipes is for instance of 40 mm.

Valves 64, 66 connect a single cold trap 60 to the sublimation chamber 30 and to the pumping system 82, 84 respectively, potentially via an interface 70.

Valves 80 connect the cold traps 72, 74, 76, 78 to a first interface 70 between the cold traps 72, 74, 76, 78 and the sublimation chamber 30, and a second interface 70 between the cold traps 72, 74, 76, 78 and the pumping system 82, 84.

The U-shaped cold traps are submerged into LN2 to reach cryogenic temperatures and trap the sublimated water and/or compounds after exiting from the sublimation chamber 30. This allows to collect the sublimated compounds, before they reach the pumping system. The cold traps have a U-shape to ensure that all the sublimated water vapour will pass in the portion of the pipe that is submerged into the LN2 bath. This ensures that all the sublimated water vapour will be collected in the cold traps, preventing water molecules from choosing an alternative straight path directly to the turbo pump 82.

The interface 70 operates the valves such that one cold trap is connected to the sublimation chamber 30 at a time during sublimation. The two valves 80 of the first U-shaped cold trap 72 are opened at the beginning. After a pre-determined duration, they are both closed, keeping the content of the cold trap 72 under vacuum, and the two valves 80 of the second U-shaped cold trap 74 are opened. The process is repeated for each cold trap. The durations can be equal or progressively increasing or decreasing, and can correspond to levels of temperature and/or time periods.

Once the sublimation process is finished, the valves 71 connecting the interfaces 70 to the sublimation chamber 30 or the pumping system 82, 84 are closed, keeping the interfaces 70 and each cold trap 60, 72, 74, 76, 78 under vacuum. The two interfacing structures 70 are vented with N2 gas to allow to detach the U-shaped cold traps, together with the HV compact fast acting gate valves 64, 66, 80 at both ends closed, and take them to a remote location while maintaining their content under vacuum.

The interfaces 70 can have one port to connect a pressure gauge and one port for alternatively venting with N2 gas or pumping out the collection system 60, 70 . . . . The port for venting with N2 gas/pumping of each interfacing structure 70 are connected by a bypass 73. As shown on FIG. 1, the same pump 15 can serve for creating a vacuum in the sealed volume 14 or in the bypass 73.

This arrangement enables to vent with N2 gas both interfacing structures 70 after the sublimation experiment is finished and when the sublimated compounds are collected into the U-shaped cold traps, while keeping the sublimation chamber, the U-shaped cold traps and the pumping system each under respective vacuum.

This is advantageous as in one embodiment, the U-shaped cold traps are disassembled with their respective HV compact fast acting gate valves 64, 66, 80 to bring them to a remote location.

The cold traps are quite wide to ensure a high conductance and in turn, a high evacuation rate from the sublimation chamber. The volume of collected water can be from a few tenths of μl or greater. It is therefore important to recover 100% of the sublimated water ice from the U-shaped cold traps and to minimize the sample exposure to ambient environment and its isotope fractionation. For this purpose, the cold trap with its two HV gate valves can be kept under vacuum after its detachment from the system and can be taken to a remote location with a transfer system 90. Keeping the U-shaped cold traps at the initial vacuum not only minimizes the sample exposure to the ambient environment but also facilitates the sample transfer to a sample tube 92.

Once at the transfer system 90, the cold trap 60 is connected to a T-shaped pipe branch, connected to a pressure gauge and a bypass. The HV valve 66 is connected to a 4-way cross. That cross is also connected to the afore-mentioned bypass, to a small glass sample tube 92 and to a valve for venting the space of the sample tube independently. The bypass is connected to a primary pump and to both sides of the cold trap 60. This system allows to pump out the volume connected to the pressure gauge (left) and to the sample tube (right) 92 to around 10⁻² to 10⁻³ mbar prior to opening the valves 64, 66. Thus, the humidity is evacuated, ensuring the transfer of 100% of the water from the U-shaped cold trap 60 to the sample tube 92 without the need to build a heating jacket that covers the whole system.

The cold trap 60 is introduced in an oil bath at a pre-determined temperature and the glass sample tube 92 is put in a LN2 bath. These bathes create a difference in pressures between the U-shaped cold trap (higher pressure) and the glass sample tube (lower pressure), which enables the transfer of water to its destination tube 92. Once the transfer is complete, the valve 66 is closed and the glass sample tube 92 is removed from the LN2 bath and closed with a cap.

The tube 92 can be a cylindrical glass sample tube with a round bottom and an upper KF flange. The round bottom facilitates the concentration of the water and its complete recovery. Once the glass sample tube 92 is detached from the system and closed, it can be further handled for analysis. For example, it can be introduced in a centrifuge at 20° C. and 1000 rpm for 10 minutes to concentrate the water in its bottom. Then, the water can be collected with a pipette with hydrophobic tips and transferred to the vial where it is being measured.

As one of the purposes of the sublimation system can be to simulate as closely as possible the lunar environment, it is important to provide a pumping system 82, 84 which enables to reach a vacuum that is as high as possible. On the Moon, the pressure ranges from 10⁻⁹ to 10⁻¹² mbar. Obtaining this amount of vacuum is very challenging for any terrestrial experimental set-up. Current experimental devices have reached a base pressure in the sublimation chamber of around 10⁻⁶ mbar. The present invention has shown to allow a pressure in the sublimation chamber 30 as low as 10⁻⁸ mbar when the thermal management system 1 and the sample holder 10 are set at −168° C.

When taking samples from the Moon surface, all sublimated water ice will be vanished into the vacuum. Hence, the sublimation system can aim at reaching a high evacuation rate (effective pumping speed) out of the sublimation chamber 30.

Pumps 82, 84 are thus constituted by a turbo pump 82 and a primary pump 84. They aim at pumping out quickly in the range of 10⁻⁶ mbar. The effective pumping speed perceived in the sublimation chamber needs to be at least equal to the maximum sublimation rate of water for the maximum temperature to be considered. Thus, the parts constituting the collection system are designed with a maximum conductance. Likewise, the crossroads between sublimation chamber/collection system/pumping system are optimized. Hence, the design of the diameters, seals, pumps, etc. is selected for ensuring the high evacuation rate needed for this system.

FIG. 2 shows an example of an embodiment of the thermal management system 1 of the invention. The same numbers refer to the same parts as presented on FIG. 1.

The thermal source can be a LN2 Dewar to which is coupled a cold rod 4. The rod 4 has a central hole that receives a heating wire 16. The rod 4 can have a first end (upper part when arranged vertically) that is generally conical and a second end (lower part) that is generally cylindrical. The shield 6 is tubular.

The shield 6 surrounds the sample in the sense that it creates a volume that is at least partially closed around the sample.

At the bottom of FIG. 2, a sample holder 10 is shown. This holder 10 can be snapped in the shield 6. This enables the retractation of the rod 40 to ensure that the sample holder is only touching the shield 6 and is thus only exchanging heat with the shield 6 on the one hand and with the sample 8 on the other hand, during a heating process.

FIG. 3 shows a detailed example of a sample holding system with a sample holder 10. The sample holder 10 can have an external peripheral groove 10.1 aimed at engaging corresponding features of the shield 6, such as leaf springs with rubies, fingers, flanges, etc.

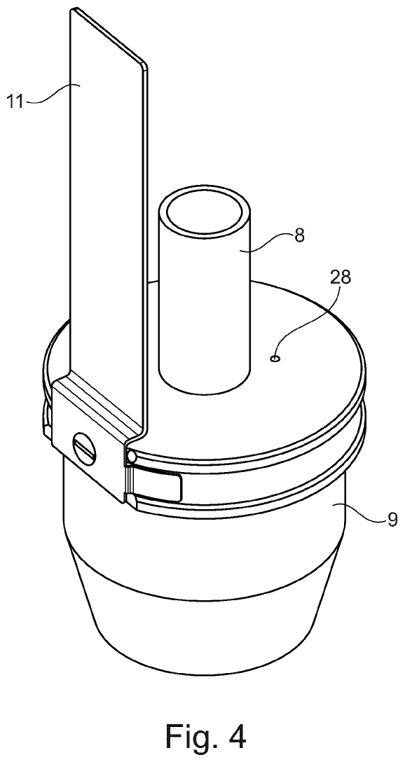

The sample holder 10 has an upper portion 10.2 with a recess (e.g., drilled hole) to receive the sample tube (see FIG. 4). The recess can have several, progressively narrowing diameters, so as to receive various diameters of sample tubes or sample adapters.

The holder 10 can be made of gold plated CuBe2. In use, the temperature distribution within the sample tube holder is homogeneous.

The sample tube holder 10 can have an integrated pt100 temperature sensor. Additionally, it can have two thermocouples, which can be introduced through a groove inside the tubular shield 6 to measure the temperature inside of the sample, in the sample tube 8, in the adapter, or where it could be required within the inner volume of the tubular shield 6. The controller 24 can also answer to those temperature measurements to regulate the temperature gradient along the whole assembly (tubular shield, sample holder, adapter, sample container and sample).

The holder 10 also has a lower portion 10.3 which can form a female connector of a bayonet coupling 42, while a male connector 44 is connected to the transfer device 40 (e.g., rod). A pair of opposite studs 44.1 of the male connector 44 engage a pair of grooves 10.31 of the female connector 10.3. A thermal insulator element 41 can be interposed between the male connector 44 and the transfer device 40.

The bayonet coupling 42 enables to releasably couple the sample holder 10 to the transfer rod 40. Thus, the sample 8 and sample holder 10 are moved by the transfer rod 40 and once the sample holder 10 is coupled to the shield 6, the transfer rod 40 can be retracted.

In one embodiment, the transfer rod will not be fully retracted because the temperature sensors are connected to the controller through the rod. Nevertheless, the bayonet coupler 42 will be detached and the transfer rod retracted a few centimetres to avoid the heat transfer between the sample tube holder and the bayonet coupler 42. It is also possible to carry out experiments where the temperature in the sample holding and transfer device will not be measured. In that case, the temperature sensors will be disconnected, and the sample tube holder 10 can be attached to the tubular shield 6, the transfer rod fully retracted out of the sublimation chamber 30.

The bayonet coupler 42 can be made of stainless steel. A thermal insulator (i.e., PEEK) can be arranged between the bayonet coupler 42 and the holder 10.

A thermal sensor 27 can be arranged on the holder 10.

FIG. 4 shows an exemplary detailed embodiment of a sample. The sample can be constituted by a sample tube containing the sample 8, held by an adapter 9 which is adapted to engage the recess of the holder 10.

The sample tube 8 can be made of quartz. The thickness of the wall can be of about 0.4 mm. It is introduced into an opening in the sample tube adapter 9, whose diameter matches the outer diameter of the sample tube 8.

Silver paint can be applied between the sample tube adapter 9 and the sample tube 8 and can dry before introducing the sample.

The sample adapter 9 can follow the same preparation procedure as the sample tube 8 before experimentation. For example, both can be transported, together with the sample, in a closed container filled with LN2. Since quartz has a low thermal expansion coefficient, temperature induced volume change can be considered negligible for the wide range of temperature changes considered for a sublimation experiment. Therefore, the sample tube 8 will not break due to the mechanical stress between the metallic sample holder 10 and the quartz tube caused by thermal expansion. Likewise, since quartz is a bad thermal conductor, the sample is expected to heat more homogeneously when the system is heated. When the sample is being heated, its environment (including tubular shield 6 and sample tube holder 10) will already have a stable temperature and the heat is transmitted by conduction from the tube holder 10 to the tube adapter 9 and to the quartz tube 8. Heat will come to the sample in a slow and accommodating manner. Additionally, the quartz will allow to perform some optical measurements when needed through a quartz view port, which can be placed in the vacuum chamber.

A Teflon or quartz frit filter can cover the top of the tube so as to prevent any solid material escaping the tube.

In case a faster sample heating is required, the quartz sample tube can be substituted by a gold-plated Cu (O free) sample tube. This can be an excellent thermal conductor and the gold plating can make the metallic surface more inert and less likely to adsorb water. This design can ensure the absence of thermal gradients when the length of the sample tube is greater. Thermal simulations have shown that for a quartz tube with 0.5 mm wall thickness, 40 mm length and 6 mm diameter, a gradient of less than 1° C. can be expected along the tube.

The sample tube adapter 9 can be made of molybdenum. Molybdenum has a low thermal expansion coefficient, preventing breakage of the tube under wide variations of temperature.

A thermal sensor 28 can be arranged on the adapter 9.

A gold-plated Cu (O free) shielding sheet 11 is provided onto the sample tube adapter 9 and placed in front of a tubular shield's 6 aperture to protect the sample tube 8 from radiative heat transfer from the environment. The height of the sheet 11 is at least equal to the height of the sample tube 8.

FIG. 5 shows the respective positions of the shield 6 and the sample holder 10 before coupling. In this example, the shield 6 is substantially tubular, i.e., defining an internal cavity for receiving the sample. The first end surface 6.11 of the shield 6 is not hollow.

The shield 6 has features 6.3 at its second end 6.2 which can cooperate with the sample holder 10 and more specifically leaf springs with rubies which cooperate with the groove 10.1 of the sample holder 10. The leaf springs 6.3 can be arranged inside the shield 6 as shown in the cross-section on the right-hand side of FIG. 5 or alternatively outside of the shield 6.3.

The shield 6 has an aperture 6.4 for the evacuation of gas. When inserted in the tube, the radiation shield 11 is such that it protects the sample tube from any radiation passing through the aperture 6.4.

1.-49. (canceled)

50. A system for sublimating a sample under high to ultra-high vacuum and at low to cryogenic temperature, and for collecting or in-line analyzing sublimated compounds thereby obtained, the system comprising:
a sublimation chamber adapted to receive the sample under high to ultra-high vacuum and at low to cryogenic temperature and provided with an outlet for evacuation of sublimated compounds;
a collection system and/or an in-line analyzing system coupled to the outlet of the sublimation chamber;
a pumping system configurated to convey the sublimated compounds from the sublimation chamber to the collection system and/or to the in-line analyzing system;
a thermal management system comprising a shield protruding into the sublimation chamber and configured to at least partially surround the sample so as to exchange heat with the sample;
a manipulation chamber connectable to the sublimation chamber; and
a transfer device for releasably handling a sample holder from the manipulation chamber to the sublimation chamber, so that the sample holder can engage the shield.

51. The system according to claim 50,
wherein the transfer device is a transfer rod for handling the sample holder, wherein the transfer device is configured to move between a retracted position and an inserted position, wherein the sample holder engages the shield when the transfer rod is in its inserted position; and
wherein a thermal insulator element is provided to thermally isolate the sample holder from the transfer device, the thermal insulator element being optionally coupled to the sample holder by a bayonet coupler.

52. The system according to claim 50, wherein the manipulation chamber is positioned relative to the sublimation chamber such that the sample holder can sit in the manipulation chamber when the transfer device is in its retracted position.

53. The system according to claim 50, wherein the manipulation chamber comprises at least one of:
a port for connection with the sublimation chamber;
at least one port for a pressure gauge;
at least one port for electrical feedthrough;
at least a port for a feedthrough, through which the transfer device protrudes;
at least one port for sample introduction, for instance a fast entry door, on which a vacuum view window or a optically transparent window can be mounted;
at least one port for dry air/nitrogen gas introduction;
at least one port for connecting a pumping system for putting the manipulation chamber under high vacuum; and
at least one port for further coupling a transfer suitcase.

54. The system according to claim 50, wherein the sublimation chamber comprises at least one of:
a port for the thermal management system;
at least one port for a pressure gauge;
at least one port for electrical feedthrough;
at least one port for sample introduction;
at least one vacuum view window or an optically transparent window; and
a port for connecting a pumping system for pumping out directly the sublimation chamber.

55. The system according to claim 50, wherein the collection system and the pumping system are configured to ensure a high evacuation rate in the sublimation chamber, so as to collect all the sublimated compounds.

56. The system according to claim 50, wherein the collection system comprises at least one port for independently venting the collection system, for instance with dry air or nitrogen gas; and/or at least one port for connecting to the pumping system.

57. The system according to claim 50, wherein the sample holder is removably coupled to the shield, such that the sample holder exchanges heat between the shield and the sample, the sample holder being provided with a peripheral groove for a snap-in and thermal coupling to the shield.

58. The system according to claim 50, further comprising an adapter inserted into a recess of the sample holder.

59. The system according to claim 50, further comprising a radiation shield mounted on the adapter or sample holder for protecting the sample from radiative heat transfer.

60. The system according to claim 50, wherein the sample holder comprises at least one temperature sensor configured to measure the temperature of the sample holder and/or of the sample and/or in the volume delimited by the shield and the sample holder and surrounding the sample.

61. The system according to claim 50, wherein the shield comprises an aperture to enable the evacuation of gases from within the shield into the sublimation chamber, the system being provided with a radiation shield protecting the sample from radiative heat transfer through the aperture.

62. The system according to claim 50, wherein the sample holder is removably connected to the transfer device by a bayonet coupler.

63. A system for sublimating a sample under high to ultra-high vacuum and at low to cryogenic temperature, and for collecting or in-line analyzing sublimated compounds thereby obtained, the system comprising:
a sublimation chamber adapted to receive the sample under high to ultra-high vacuum and at low to cryogenic temperature and provided with an outlet for evacuation of sublimated compounds;
a collection system and/or an in-line analyzing system coupled to the outlet of the sublimation chamber;
a pumping system configurated to convey the sublimated compounds from the sublimation chamber to the collection system and/or to the in-line analyzing system;
a thermal management system comprising a shield protruding into the sublimation chamber and configured to at least partially surround the sample so as to exchange heat with the sample.

64. The system according to claim 63, wherein the collection system comprises a cold trap fluidly connectable to the outlet of the sublimation chamber, the cold trap being preferably constituted of a U-shape pipe the outer surface of which being partly immerged into a cold or cryogenic medium such as LN2.

65. The system according to claim 64, wherein the cold trap comprises a first end connectable to the outlet of the sublimation chamber, potentially via an interfacing structure enabling or preventing the fluidic connection between the sublimation chamber and the cold trap, and a second end connectable to the pumping system potentially via an interfacing structure, and wherein the cold trap, the optional interfacing structures and the sublimation chamber are optionally configured to be maintained under a dynamic high to ultra-high vacuum.

66. The system according to 64, wherein the cold trap comprises two gate valves respectively at the first and second end of the cold trap.

67. The system according to claim 64, wherein the cold trap is adapted to be disconnected from the sublimation chamber and to be sealed so as to maintain the sublimated compounds under vacuum.

68. The system according to claim 63, wherein the collection system and the pumping system are configured to ensure a high evacuation rate in the sublimation chamber, so as to collect all the sublimated compounds.

69. The system according to claim 63, further comprising:
a transfer device, for instance a transfer rod for handling the sample or a sample holder, wherein the transfer device is configured to move between a retracted position and an inserted position, wherein optionally the sample holder engages the shield when the transfer rod is in its inserted position; and
a thermal insulator element provided to thermally isolate the sample or sample holder from the transfer device, the thermal insulator element being coupled to the sample or sample holder by a bayonet coupler.
